(19) United States
(12) Patent Application Publication
(10) Pub. No.: US 2023/0304959 A1
(43) Pub. Date: Sep. 28, 2023

(54) SOLID STATE SOIL SENSOR

(71) Applicant: Plant Bioscience Limited, Norwich (GB)

(72) Inventors: Tony MILLER, Norwich (GB); Yi CHEN, Norwich (GB); Martin PEACOCK, Royston (GB)

(21) Appl. No.: 18/008,003

(22) PCT Filed: Jun. 3, 2021
(86) PCT No.: PCT/EP2021/064939

§ 371 Date: Dec. 2, 2022

(30) Foreign Application Priority Data
Jun. 5, 2020 (GB) 2008507.2

(51) Int. Cl. G01N 27/333; G01N 33/24

(52) U.S. Cl. CPC G01N 27/3335; G01N 33/24

(57) ABSTRACT

A solid-state soil nutrient sensor comprising a sensor blade for inserting into the soil, the sensor blade comprising an electrically insulating substrate. The sensor may further comprise first and second electrodes disposed on the substrate, each electrode comprising: a sensing region located towards an end of the sensor blade inserted into the soil, and a contact region displaced away from the end of the sensor blade and electrically connected to the sensing region, for making an electrical connection to the electrode. The sensor may further comprise electrical insulation over each of the first and second electrodes between the sensing region and the contact region; a reference membrane over the sensing region of the first electrode; and a nutrient sensing membrane over the sensing region of the second electrode; and the reference and nutrient sensing membrane each comprise one or more layers of solvent-cast polymer.

### TECHNICAL FIELD

This invention relates to solid state soil chemistry sensors, for sensing concentrations of soil water nutrients, and to the fabrication and use of such solid-state sensors.

### BACKGROUND

Nitrogen-based fertilisers are used ubiquitously in agriculture, and effective management of the amount of fertiliser used is important. The volume of fertilisers applied to land may be estimated based only on the experience of the grower or farmer. In general, it is important to manage economic efficiency and environmental management, as it is known that excess nitrogen run-off from agriculture can enter the water supply, which is a cause for concern for humans and marine eco-systems. For example, the run-off contamination of water supplies can cause a proliferation of microorganisms, which leads to de-oxygenation of the water and the death of fish.

Various techniques are known for monitoring nitrate levels in soil. Typically, a soil core is taken, the nitrogen is extracted, and this is then used to predict the amount of nitrogen available to the roots of plants in the soil. However, this is a time consuming process and may take weeks to yield results. This procedure is cumbersome, slow and expensive and there is a need for more automated, efficient, and inexpensive techniques. Additionally, when the testing of a soil sample takes place at a remote lab there is a challenge of correlating the lab’s test results back to the specific point of collection. Moreover, soil-core methods provide only a snapshot of the nitrate levels in soil.

In addition to soil testing in discrete locations, crops may be remotely monitored by cameras/spectrometers attached to satellites, drones, farm vehicles and the like. However, these methods utilize imaging and spectral technology merely to interpret the health of the already-grown plant tissue. Therefore, an undesirable soil-chemistry composition may be identified too late, and only at the point where a plant’s health is adversely affected. Thus, it is often too late to take remediation action to improve soil composition.

Therefore, a way to robustly measure a continuous concentration of nutrient levels in soil is desirable.

Regarding the general technical field, known documents include: EP 3537153 A1; WO 2009/049093; JP 2013/231644; and Sensors and Actuators: Chemical, Vol. 88 (3), Artigas J., et al “Development of a screen-printed thick-film nitrate sensor based on a graphite-epoxy composite for agricultural applications”.

### SUMMARY

Aspects and preferred features are set out in the accompanying claims.

The present disclosure relates to a solid-state soil nutrient or nitrate sensor.

According to one aspect of the present invention, there is provided a sensor that comprises: a sensor blade for inserting into the soil, the sensor blade comprising an electrically insulating substrate and first and second electrodes deposited on the substrate. Each electrode comprises a sensing region located towards an end of the sensor blade inserted into the soil, and a contact region displaced away from the end of the sensor blade and electrically connected to the sensing region, for making an electrical connection to the electrode. The sensor further comprises electrical insulation over each of the first and second electrodes between the sensing region and the contact region; a reference membrane over the sensing region of the first electrode; and a nitrate sensing membrane over the sensing region of the second electrode. The sensing regions of the first and second electrodes are less than 10 mm apart on the sensor blade; and the reference membrane and the nitrate sensing membrane each comprise one or more layers of solvent-cast polymer.

The electrodes disposed or deposited on the substrate may be carbon or silver chloride electrodes. Alternatively, the electrodes can comprise other materials, for example: platinum, gold, and other chemically inert conductive materials. Manufacturing techniques also applicable include, dip coating, spray coating, screen printing, digital drop deposition, and the like.

Both the insulating substrate and membranes are preferably deformable, or have a degree of plasticity, such that they are able to withstand being repeatedly inserted into soil. Preferably, the sensor membranes are disposed in two or more layers such that the membranes are thicker, more robust, and therefore have improved reusability. Moreover, it is beneficial for the sensor’s lifespan to provide relatively thick and/or multiple layers which are resistant to the abrasive forces of being repeatedly inserted into soil.

It is further advantageous to maintain an electrode separation at or below 10 mm in order that the sensor is sensitive even in dry soils. The electrical conductivity of the soil is dependent on the ionic strength of soil water and therefore the moisture content of the soil, the relationship is complex, however soil is generally less conductive when dry. It will be understood that electrodes referred to as silver chloride electrodes may also comprise metallic sliver, i.e., the electrodes may comprise a mixture of Ag and AgCl. The reference electrode is an inert electrode which provides a fixed reference point when making electrical contact with the soil; it provides the reference measuring point relative to the second ion-selective electrode, against which a voltage is generated. The base substrate for the two electrodes in general may comprise any suitable electrically insulating material, for example plastic or ceramic.

The nitrate sensing membrane may comprise a nitrate-ion selective membrane. The nitrate-ion selective membrane is present only in the sensing membrane, and not in the reference membrane. Furthermore, the nitrate sensing membrane may comprise a nitrate-sensing reagent to convert nitrate ions to electrons, or a sensed voltage. More specifically, the sensing membrane may comprise a nitrate-sensing reagent to convert changes in the concentration of nitrate ions to changes in voltage. Thus, in some examples, nitrate ions in the soil do not transverse through the membrane directly; instead, nitrate ions may occupy nitrate binding centers within the nitrate-selective membrane.

The nitrate sensing membrane may comprise a plasticiser, a polymer, and a nitrate-ion selective reagent. Merely for example, the plasticiser may be a suitable alkyl ether, and the polymer may be PVC. Additionally, the nitrate sensing membrane may comprise a charge balancing lipophilic ion. It is advantageous to form the membrane with a plasticiser in order to provide the membrane with the physical property of plasticity. In other words, it is advantageous for the membranes to have a relative amount of flexibility or plasticity such that, in use, the fabricated sensors are more resilient to bending forces and being repeatedly inserted into soil.

The nitrate sensing membrane may comprise two layers of solvent-cast polymer, where an inner layer comprises the nitrate-ion selective membrane or nitrate-sensing reagent, and an outer less-selective layer that is physically harder than the inner layer. Thus, in some examples, the rigidity or hardness of an outer layer of the membrane may be increased by using a different membrane composition. For example, a relatively higher proportion of plasticizer may be used. Overall, a harder outer layer provides the benefit of creating membranes that are more resistant to abrasion in use.

The reference membrane may comprise a charge-selective membrane that allows positive charge to flow through the reference membrane and inhibits movement of chloride ions through the reference membrane. For example, membranes may comprise ionomer such as nafion, which provides a barrier to the transport of chloride ions. Further, the reference membrane may not be ion-selective. The reference membrane may allow charge to flow through the reference membrane without the movement of ions through the reference membrane. For example, membranes may comprise an ionomer such as nafion, which provides a barrier to the transport of chloride ions. In general, the reference membrane may selectively allow charge to flow through the membrane, and inhibit the movement of ions (i.e. charged molecules) through the membrane.

The reference membrane may comprise two layers of solvent-cast polymer: an inner layer comprising the charge-selective membrane, and an outer less-selective layer, wherein the outer less-selective layer is harder than the inner layer.

Both electrodes may be carbon electrodes, or both may be silver chloride electrodes. Alternatively, the two electrodes may comprise different materials: for example, the electrode disposed underneath the sensing membrane may comprise carbon, and the electrode disposed underneath the reference membrane may comprise silver/silver chloride.

According to another aspect of the present disclosure, there is provided a method of fabricating the solid-state soil nitrate sensor as defined above. The fabrication method comprises: screen printing the first and second carbon or silver chloride electrodes onto the substrate; solvent casting one or more layers of polymer over the sensing region of the first carbon or silver chloride electrode to form the reference membrane; and solvent casting one or more layers of polymer over the sensing region of the second carbon or silver chloride electrode to form the nitrate sensing membrane.

It will be understood that the process of solvent casting generally comprises drawing off the solvent to leave behind a solid-phase cast of the formerly solvated materials. The solvent can be allowed to dry in room-temperature air to promote a homogenous membrane with an even surface. Alternatively, other accelerated drying methods can be used, including one or more of: vacuum chambers, desiccators, and drying ovens. For larger scale production, it may be most efficient to accelerate the drying process using desiccators, for example. It is advantageous to use a relatively slow-drying solvent when solvent-casting by screen-printing, because less-volatile solvents generally work better in screen-printing apparatuses, e.g., are less likely become viscous and clog the printing apparatus.

Nevertheless, it will be apparent to the skilled person that screen-printing can be replaced by a manual, or automatic, pipetting process. In these examples, a relatively fast-drying solvent may be used in order to speed up the solvent-casting drying step.

The method may also comprise, when forming the solutions for screen-printing, dissolving a lipophilic plasticiser, a polymer, and a nitrate-ion selective reagent in a solvent to form a membrane solution, and screen-printing a volume of the membrane solution over the sensing region of the second carbon or silver chloride electrode.

The solvent may be a ketone with a relatively higher boiling point e.g. a boiling point higher than water in standard temperature and pressure conditions. This is beneficial for screen-printing because such ketones are relatively slow-drying solvents. In other fabrication methods, it may also be advantageous to use a relatively faster-drying solvent, such as tetrahydrofuran (THF).

According to another aspect of the present disclosure, there is provided a solid-state sensor for in-situ sensing of soil nutrients. The solid-state sensor comprises an electrically insulating substrate bearing a first and second electrically conductive track; a contact region defined by electrical contacts at an end of each electrically conductive track; a sensing membrane disposed over a second end first electrically conductive track, defining an ion-selective electrode; a reference membrane disposed over a second end of the second electrically conductive track defining a reference electrode; an electrically insulating cover disposed over the first and second electrically conductive tracks. Each of the sensing membrane and the reference membrane comprises a layer of solvent cast polymer, and the sensing membrane comprises a sensing reagent for sensing a specific nutrient. A separation between the second end of each electrically conductive track is less than around 10 mm. The substrate in general may comprise plastic or ceramic. The substrate is preferably comprised of a flexible/deformable polymer.

The sensing reagent may be for sensing a concentration of nitrate. However, the sensing reagent may also be for sensing a plurality of alternative soil-based nutrients chosen from any one of: sodium, potassium, phosphate, ammonium, calcium, protons (i.e., a pH sensor). Yet further, the sensing membrane may comprise a sensing reagent that is specific to sensing a soil contaminant, for example cadmium or lead.

The sensing membrane and the reference membrane may comprises two layers of solvent-cast polymer. Multiple membrane layers create more robust membranes, which in turn increases their reusability. In other words, it is beneficial for the sensor’s lifespan to provide relatively thick and/or multiple layers which are resistant to the abrasive forces of being repeatedly inserted into soil.

Each of the first and second electrically conductive tracks may comprise carbon or silver chloride, and substantially all of the first and second electrically conductive tracks may be covered by the electrically insulating cover. The electrically insulating cover provides waterproofing.

Each of the first and second electrically conductive tracks may be formed by screen-printing onto the substrate. The electrical contacts are preferably integrally formed as part of the first and second electrically conductive tracks, and are screen-printed as part of the same screen-printing step that forms the conductive tracks. In other words, the contact region, tracks, and electrodes underlying the membranes may be printed with the same material in a single screen-printing step.

Each of the sensing membrane and the reference membrane may contain the same polymer, for example polyvinylchloride (PVC), preferably high molecular weight PVC.

According to any of the above described solid state nutrient or nitrate-specific sensors, the sensors may comprise a voltage sensor, or voltage reading electronics, coupled to the contact region, and a wireless network transmitter coupled to said voltage sensor to enable wireless collection of soil chemistry data from said soil chemistry sensor.

Generally, the electrical contacts or contact regions may be suitable for coupling with an external data logging device. The external data logging device may serve the purpose of both collecting and recording signal-data provided by the device. Furthermore, the data-logging device logs the output voltage from the sensor it does not apply a voltage to the sensors.

The external data logging device may be configured to measure an impedance value indicative of a concentration of nutrient in a soil sample. Furthermore, the external data logging may be further configured to measure a resistance, current, or conductance value and the like, indicative of a concentration of nutrient in a soil sample. The external data logging device may further be configured to infer a soil-moisture content from a conductance, impedance, admittance, capacitance measurement from the sensor, as the concentration of water and the ionic strength of the water held in the soil affects the conductivity of the soil.

Additionally or alternatively, the solid-state sensor may comprise moisture sensing means, which may simply be computer-programmable code stored on a memory of the data logger, designed to be executed on a processor to infer a moisture level. Alternatively, the moisture sensing means may be a separate moisture-sensor disposed on the sensor itself and configured to make independent soil-moisture measurements. As a further alternative, some examples of the sensor or sensor blade may contain two counter/reference electrodes in addition to a sensing (ion-selective) electrode. In these examples, such a measurement of soil-moisture content can be determined from a conductivity measurement taken between the two reference membrane electrodes, when placed in the soil. Thus, the external data logger may be configured to correct a measured concentration of nutrient or nitrate in the soil, the correction based on a measured soil-moisture content level.

Yet further, the sensor or sensor blade may have temperature sensing means disposed on it, which in turn may be coupled to the data logger for recording soil temperatures. For example, the substrate may have deposited on it a digital thermometer, such as a semiconducting resistor or a thermistor, which can measure the temperature of the soil independently of the nutrient-sensing electrodes.

According to a further aspect of the present disclosure, there is provided a system for continuous in-situ sensing of soil nutrients comprising a plurality of solid-state sensors as defined in any of the above examples. The system may comprise a plurality of signal amplifiers, one for each solid-state sensor, wherein each signal amplifier is communicatively coupled to a data logger for receiving signals from each solid-state sensor. Advantageously, by providing a plurality of nutrient sensors in a plurality of lateral locations over a plot of land, and/or at a variety of depths, it is possible to simultaneously measure a special profile or distribution of soil-nutrient concentrations, over a period of time.

### Additional Examples

The electrodes disposed or deposited on the substrate are preferably silver/silver chloride electrodes. Electrodes referred to herein as silver chloride, or silver/silver chloride electrodes, may comprise metallic silver, e.g. a mixture of Ag and AgCl. Further manufacturing techniques for the electrodes includes providing a printed circuit board (PCB).

It will be understood that, in examples, the nitrate sensing membrane comprising a nitrate-sensing reagent is to sense nitrate ions. In embodiments, this sensing may comprise converting nitrate ions into electrons.

Either or both of the first and second electrodes may generally be formed from any one of: carbon, silver, sliver chloride, gold, or platinum. Furthermore, the first and second electrodes may be first and second carbon electrodes, silver/silver chloride electrodes, copper/copper sulphate electrodes, or generally any suitable redox electrode. These materials are generally compatible with the manufacturing techniques described above.

A solid-state soil nitrate sensor according to any of the above-described examples may be manufactured by printing the first and second electrodes onto a printed circuit board (PCB). This manufacturing method may further comprise: solvent casting one or more layers of polymer over the sensing region of the first electrode to form the reference membrane; and solvent casting one or more layers of polymer over the sensing region of the second electrode to form the nitrate sensing membrane.

### BRIEF DESCRIPTION OF THE DRAWINGS

Examples of the invention will now be further described with reference to the accompanying figures in which:

FIG. 1 shows a plan view of a sensor blade with a solid-state sensing region;

FIGS. 2a and 2b show a cross-sectional view through the sensing region of the blade shown in FIG. 1, and show the sensing and reference membranes, respectively;

FIG. 3 shows a sensor system incorporating multiple soil chemistry sensors of the type shown in FIG. 1;

FIG. 4 graphically illustrates a sensor system as shown in FIG. 3;

FIG. 5 shows an embodiment of a probe housing system housing a plurality of sensors, suitable for measuring soil nutrients at a plurality of depths; and

FIG. 6 illustrates an example of the probe housing as shown in FIG. 5 when placed in soil, and further illustrates the internal locations of the sensors.

### DETAILED DESCRIPTION

Ion-selective membrane (ISM)-based sensors, which use ion-selective electrodes (ISEs), are known in the art and provide a promising approach to detecting soil nutrients. However, ISM-based sensors often require an inner filling solution between the ISM and a conductive metal layer substrate used to create electrical contact. This creates potential drawbacks such as: contamination of the solution, gradual evaporation of the solution (e.g., during storage or pronged use) which alters the sensitivity of the sensor, and delamination and poor adhesion of the sensing/reference membrane. Specifically, the inner filling solution can leave the ISM sensor and permeate the surrounding dry soil, which thus alters the concentration of the inner filling solution and affects the sensor output voltage. Moreover, it is difficult to fabricate sensors having an inner solution filing with a small form-factor.

In the present disclosure, the inventors have identified a solid-state nutrient sensor that is able to mitigate the drawbacks associated with solution-filled ISM-based sensors. For example, solid-state sensing elements may improve both the sensing element’s robustness, and shelf life, given that the content of a solid-state membrane cannot be lost by evaporation. Furthermore, the solid-state sensing sensor blade as described in this disclosure is a planar (two-dimensional) device, which may be produced by mass-production techniques such as screen-printing.

Generally speaking, references to solid-state in the present disclosure refer to the final state of the membranes present in the sensing region of the device. Solid-state encompasses phases of any of crystalline, semi-crystalline, amorphous-solid and polymeric structures, where it will be understood that these phases are not mutually exclusive to one another.

To increase the efficiency of fertilizer application it is necessary to have an in-situ real-time continuous measurement of nutrients: for example, nitrates, phosphates, and potassium. In the case of agricultural fields, the ability to apply the fertilizer when and where it is needed is based on having the ability to measure nutrient concentrations at a plurality of depths and a plurality of locations across a field or plot of land.

Consistent with this need, a plurality of the individual solid-state sensors described in the present disclosure can be used simultaneously. This enables a dynamic temporal monitoring of nutrient levels in soil, and a spatial monitoring of soil nutrient. In this way, an array of solid-state sensors may be used to monitor the directional rate of change of a soil’s nutrient composition.

The ability to measure a spatial profile of soil nutrients is advantageous, not least because fertilizers may be added to fields by spraying. The fertilizers subsequently seep into the soil to form a vertical nutrient-concentration gradient. The ions comprised in the fertilisers (e.g., potassium, nitrate, and phosphate ions), however, can undergo both reversible binding and irreversible binding with other components in soil, or may be unevenly absorbed by a random distribution of roots and microbes. Therefore, soil can develop a complex and unpredictable nutrient profile, which varies both spatially and with time. Thus, a reliable method of measuring this unpredictable distribution of soil nutrients is to use an array of soil sensors having the ability to continuously monitor nutrient concentrations.

The sensing elements of the sensors disclosed herein are electrochemical in nature. Therefore, the concentration of ions in the soil can be detected using an ion-selective formulation immobilized over a conductive surface. The conductive surface onto which the ion-selective formulation is immobilised is generally a working electrode. A reference membrane is then typically immobilized over a further conductive surface; typically a reference, or counter, electrode. In further embodiments of the device, a reference membrane is disposed over a reference electrode, a sensing (e.g. ion-selective) membrane is disposed over a working electrode, and a third, exposed, counter electrode is present.

This nutrient data may additionally be correlated with other channels of data, including soil pH, soil temperature, and soil humidity. In particular, soil moisture content affects the sensitivity and operation of the solid-state membranes. Therefore, embodiments of the solid-state device further comprise a moisture sensor to detect moisture levels in soil. The moisture levels may be used to calibrate the nutrient response of the sensor. In this way, the device may be configured to inherently take account of soil-moisture content which, in turn, can influence the effective concentration of nutrient available to a plant. Therefore, in examples, the sensing device may be configured to report an effective concentration of available nutrient, rather than the true concentration of nutrient present in the soil. For example, the sensor generally measures the soil-water nutrient concentration; therefore, as the soil dries the concentration of nutrient effectively increases.

Generally speaking, the nitrate available to a plant/crop is a function of the absolute amount of nitrate in the soil, and the humidity of the soil. A region of soil that has a high concentration of nitrate but zero water content cannot provide a plant nitrate, as the roots are not able to access the nitrate, either by osmotic or active transport mechanisms. Therefore, in use, where a soil sample has zero moisture, the sensing element described herein will measure a nitrate concentration of zero.

Alternatively, in some examples, the sensor may have an integral or external moisture sensor for simultaneously measuring the water content of the soil. For example, an external soil moisture sensor may be co-located with the nutrient sensor in order to measure an amount of soil moisture in the vicinity of the nutrient sensor. Alternatively, the sensor itself may be configured to indirectly measure the soil moisture content by way of a conductivity measurement. For example, electrodes which form the reference sensing elements of the nutrient sensor may be configured to measure a conductance or conductivity of the soil, in response to an applied voltage. The sensor’s response to the applied voltage is indicative of the ionic strength of the soil that in turn can be used to infer the soil moisture content. The soil moisture content can subsequently be used to ‘correct’ a measured soil nutrient concentration to obtain a ‘true’ value of soil nutrient concentration.

Consistent with the above, a calibration or correction step may be performed to determine the dependency between measured nutrient content and measured water content. Additionally, calibration data may simply be stored in a computer, offsite server or system that processes the data retrieved from the sensor. Therefore, in some examples, a computer or data-logger which processes a signal received from the nutrient sensor may be configured to convert a measured concentration of nutrient (i.e. an effective nutrient level, which is actually available to a plant) to a true concentration of soil nutrient. In this way, the data-logger may be configured to use both the signal measured from the sensing region of the solid-state sensor (indicative of the nutrient of interest), and the signal from the moisture sensor.

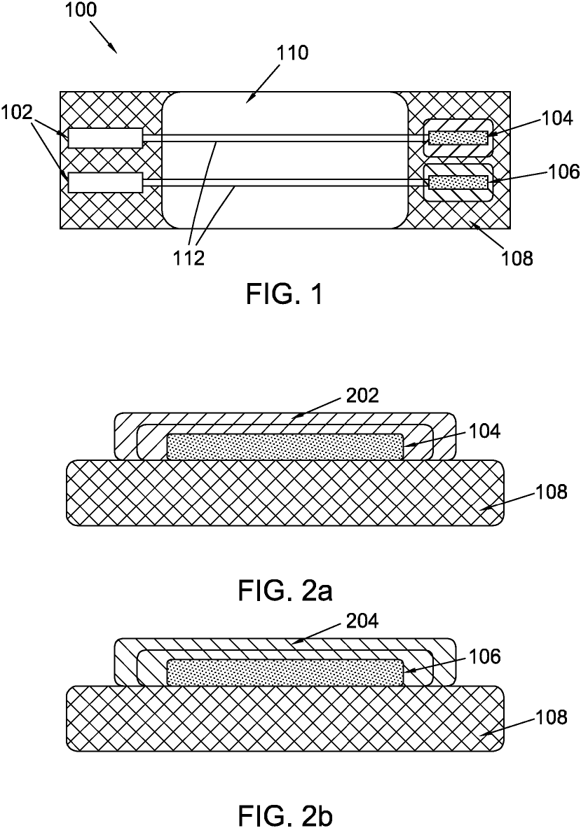

FIG. 1 shows a plan view of a sensor blade 100, disposed on an inert base 108, where the base bears a conductive track 112 covered in an electrically insulating cover 110. At the left hand side of the blade is a pair of electrical contacts that make electrical connection with a sensing electrode 106 and a reference electrode 104, via the tracks 112. Together, the sensing electrode 106 and the reference electrode 104 constitute a sensing region of the blade. The inert base 108 may comprise ceramic, polymer, or plastics in general. For example, the base may comprise polyethylene terephthalate. Preferably, the material used to make the base is also impervious/repellent to moisture, e.g. made of a hydrophobic polymer, such that any moisture in the soil is less likely to de-laminate any screen-printed aspect of the blade, in use. The base 108 may be rigid (e.g., when ceramic is used) or may be deformable, e.g. when polymers such as polyethylene terephthalate are used.

The electrically insulating cover 110 may comprise any suitable non-electrically conductive material, for example: polymer coatings such as polypropylene or epoxy resin, or rubber. Merely for example, the cover 110 may be disposed over the blade’s body as a cold-shrink tubing (i.e., a pre-stretched elastomer, which shrinks upon removal of the supporting core during application). In this example, although not shown in FIG. 1, the cold-shrunk tubing cover would envelop the blade on its top surface (shown) and its underside (not shown). Cold-shrink may be advantageous because it avoids the application of heat, which can potentially damage the membranes 104, 106.

Each of the contacts 102, tracks 112, and sensing/reference electrodes may comprise Carbon or Ag/AgCl. The electrode tracks 112 are preferably screen-printed onto the inert base 108. Furthermore, different materials may be used for each electrode track. For example, in a preferable embodiment, carbon is used for the track adjoining the sensing electrode 106, and Ag/AgCl is used to form the track adjoining the reference electrode 104. Any carbon material suitable for screen-printing may be used for the tracks 102, contacts 102, and electrodes 104, 106, for example graphene-nanoplatelet (GNP) carbon. Advantageously, screen-printed carbon electrodes (all of 102, 112, 104, 106) can be printed by mass-production onto a flexible polymer base substrate, such that the complete sensor blades 100 can be produced rapidly. In general, it will be appreciated that any electrically conducting material capable of being screen-printed can be used to form the electrode tracks 112 and/or electrodes 104, 106.

The example illustrated in FIG. 1 comprises two electrodes, where the sensing electrode 106 is covered in a nutrient-specific solid-state membrane, which is preferably an ion-selective membrane, and the reference electrode 104 is covered in a solid-state reference membrane. The membranes may be solvent-cast membranes, and may be disposed e.g. by manual/automated pipetting, or by screen printing and the like. Similarly to the base, the membranes may be fabricated to include a plasticiser to increase a flexibility of the membrane. For example, it is advantageous to match a flexibility of the base/substrate 108 to the membranes (104, 106) in order to maintain the integrity of the membranes in use and improve the lifetime of the device.

Generally speaking, the solid-state sensing element consists of two or more electrodes, comprising at least a reference electrode 104 and an ion-sensing electrode 106, which is typically a working electrode. The electrode system may further comprise a counter electrode (not shown), which may be left exposed, in addition to a reference and working electrode. Further, as mentioned above, in some examples two reference electrodes may be provided in the sensor in addition to an ion-sensing electrode, wherein the two reference electrodes can be used to infer a soil-moisture content by determining a conductivity or conductance measurement of the soil.

The working/sensing electrode 106 consists of a conductive surface, onto which is deposited a formulation comprising at least one solvent, and organic compounds (and possibly inorganic compounds). In examples where the membrane formulation is solvent-cast, the formulation is cured/dried such that the solvent evaporates, whereupon the formerly solvated organic compounds form a solid-state sensing/reference membrane layer. In use, this membrane layer is directly exposed to the soil. The sensing membrane may comprise similar organic reagents to those in the reference membrane, with the exception of the addition of a nutrient-specific compound, which may be an ion-selective compound specific to the nutrient of interest. Furthermore, the sensing membrane may contain a plasticiser not present in the reference membrane, used to give the sensing membrane a degree of plasticity. Generally, it is preferred that the formulation used to deposit the reference membrane is invariant to the nutrient of interest, and invariant with respect to the ion-selective reagent used in the sensing membrane.

The electrodes 104, 106 in the sensing region are not required to be any particular size or dimension; indeed, it can be advantageous for the electrodes 104, 106 to have a small form factor, as this can increase the robustness of the device whilst simultaneously lowering the cost of manufacture.

The sensing region (i.e., comprising electrodes 104, 106) can be viewed as an electrochemical cell. However, it is not necessary to have a formal electrolyte between the two electrodes because, in use, the sensing region is designed such that moisture and ionic compounds comprised in the soil or growing medium (into which the blade is inserted) provide sufficient electrical contact between the two electrodes. This electrical contact is improved by disposing the tips of the electrodes in the sensing region with around 10 mm of separation, or preferably less. Larger electrode separations are also possible, however, increasingly large separation above 10 mm may require increasingly wet soil for the sensor to operate effectively.

The sensor blades are configured to use negligible applied voltages or currents, including using: open circuit potential, potentiometry or chronopotentiometry to detect the nutrient of interest. In other words, the voltage or current (including impedance, conductance, and the like) produced by the sensor is measured passively. The soil-nutrient ions interact with the sensor’s sensing electrode, which produces a voltage that can be measured e.g. by a data-logger. The distortion of signal (i.e. current, resistance, impedance etc. from the sensor) is generally proportional to the product of the impedance of the (soil) sample and the current flowing through the sensor. For example, high impedance and high current can result in to high distortion, and high impedance and low current can result in a relatively lower level of distortion. As mentioned, in general, the reference electrode is an inert electrode that provides a fixed-potential reference point when making electrical contact with the soil. The reference electrode thus provides the reference potential relative to the ion-selective electrode, against which a voltage is generated.

When the concentration of an ion indicative of a nutrient of interest (merely for example, nitrate, potassium, or phosphate cation) in the soil environment decreases, the voltage across the sensor increases. This relationship between ion concentration and sensor voltage can be modelled according to a variation of the Nernst equation called the Nickolsky-Eisenman equation (which takes into account interference from ions other than the target ion):

$$E = K + \left(\frac{2.303 R T}{{z}_{i} F}\right) log \left({a}_{i} + {k}_{i j} {a}_{j} {}^{\frac{{z}_{i}}{{z}_{j}}}\right)$$

where E is the potential, zᵢ and aᵢ are the charge and activity (i.e., effective concentration) of the ion of interest, K is a constant dependent on the probe design, R is the gas constant, T is absolute temperature, F is the Faraday constant, kᵢⱼ is the selectivity coefficient. The label i denotes target ions, and j denotes the interfering ions. Thus, the selectivity coefficient, kᵢⱼ, quantifies the ability of the sensing electrode to discriminate the ion of interest i, against interfering ion j.

FIG. 2a shows a cross sectional view of the reference electrode 104 and two layers of the reference membrane 202 disposed over the electrode. Similarly, FIG. 2b shows a cross sectional view of the sensing electrode 106 and two layers of the sensing membrane 204. It is possible that, in other examples, only a single layer of each membrane is present.

In general, at least one layer of membrane is required in the sensing region, for each of the sensing and reference electrodes. In order to obtain a layer of sufficient thickness, however, the membranes may be disposed during fabrication in two solvent-casting (or screen-printing) steps, i.e. to ensure that all solvent used to dissolve the respective formulations the membranes fully evaporates. For example, it may be advantageous to prepare the membranes in two or more steps, producing two or more layers. It is nevertheless possible in some examples to deposit the membranes in a single pipetting or screen-printing step, simply using a more viscous membrane formulation, which is formed by using proportionally less solvent. There are several advantages associated with thicker sensing and reference membranes. For example, increasingly thick membranes have a longer lifetime due to an increased resistance to abrasion from being repeatedly inserted into soil.

The membrane layers are generally disposed by solvent-casting a liquid or gel formulation of organic agents dissolved in a solvent, whereupon the solvent evaporates. The evapouration can be accelerated by heating, or drying using a vacuum chamber or desiccator, or oven and the like, to leave behind a solid-phase cast of the organic agents as a membrane. The solvent casting may be carried out by techniques such as by pipetting and screen-printing, and the like. Generally, the membrane formulations are disposed directly onto the base or electrodes 104, 106 in the sensing region. Detailed examples of fabrication techniques and membrane formulations are described below.

Nevertheless, in examples where two layers of solvent-cast, or screen-printed, membranes 202, 204 are disposed, each layer (i.e., the inner and outer layer) generally has the same composition. In other words, preferably, both layers illustrated in the sensing membrane 204 in FIG. 2b share an identical composition, having been disposed in two solvent-casting steps. The same applies for the two layers of reference membrane 202. The formulations used to prepare each of the sensing and reference membranes generally comprise at least one polymer, which is dissolved in a solvent before solvent-casting. Alternatively, an outer layer membrane may be formed using a different composition having a relatively higher proportion of a hardening reagent, which creates a harder outer layer relative to the inner layer.

In embodiments, it may be beneficial for each membrane to contain the same polymer, for example a matrix of polyvinyl chloride (PVC). The addition of an ion-selective component is exclusive to the sensing membrane, where the ion-selective component is specific to an ion representative of the nutrient of interest in the soil.

Furthermore, the reference membrane formulation may optionally contain a charge-selective membrane that allows charge to flow through the reference membrane, and inhibits movement of negatively charged ions, such as chloride ions, through the reference membrane. High chloride ion concentrations can interfere with the nitrate-selective membranes, which may result in an erroneous measurement of soil-nitrate concentration by the sensor at low soil nitrate concentrations. However, it will be appreciated that the reference membrane itself is not ion-selective, and is insensitive to changes in soil’s ion concentration. Thus, it is advantageous in some examples to provide such a positive-charge selective layer, for example nafion or other suitable ionomer, in the reference membrane layer.

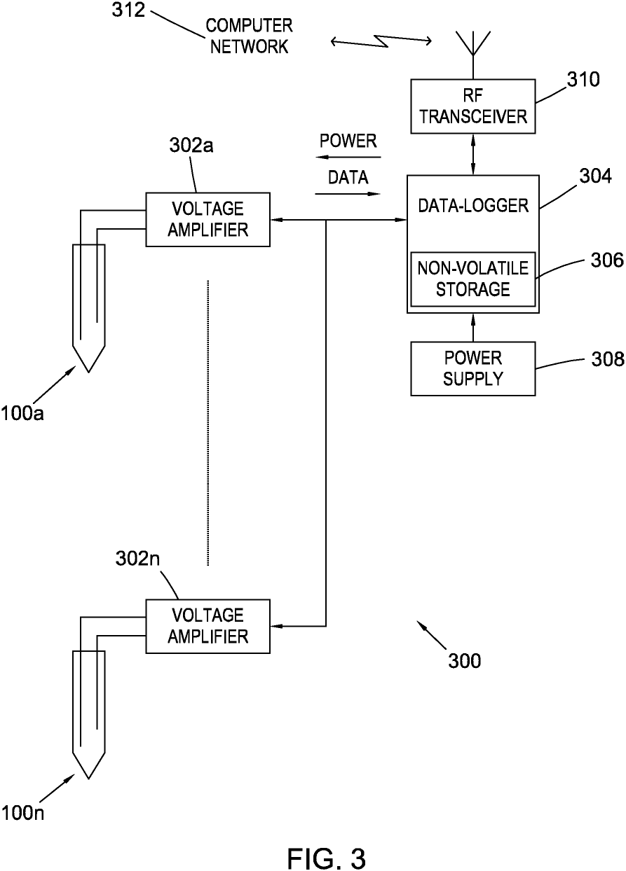

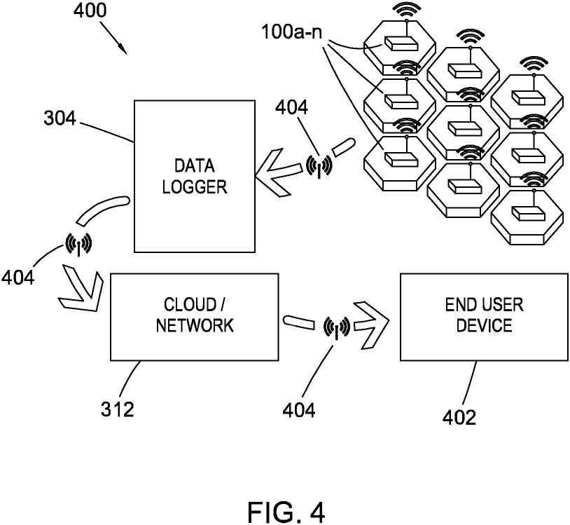

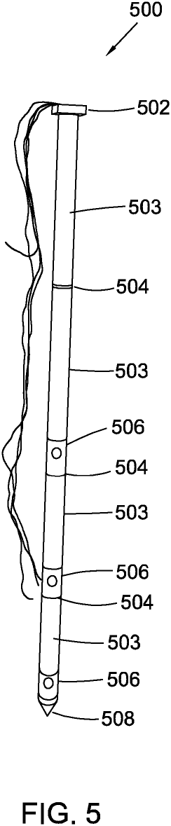

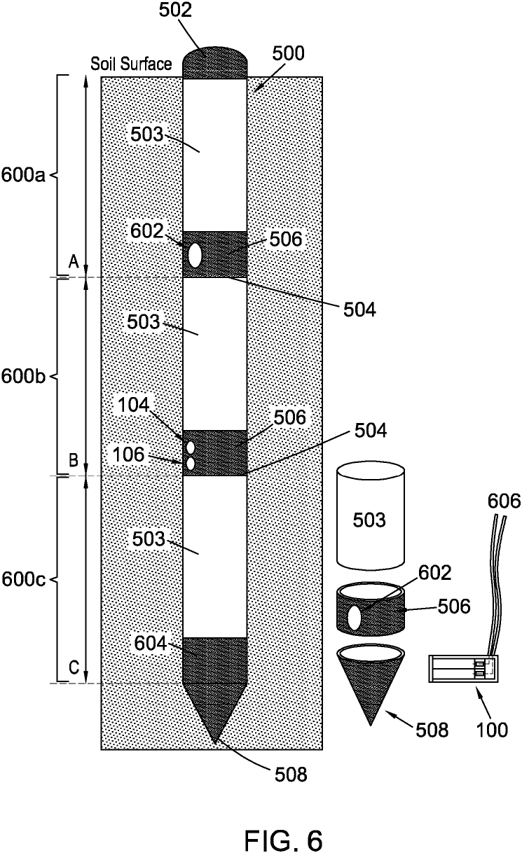

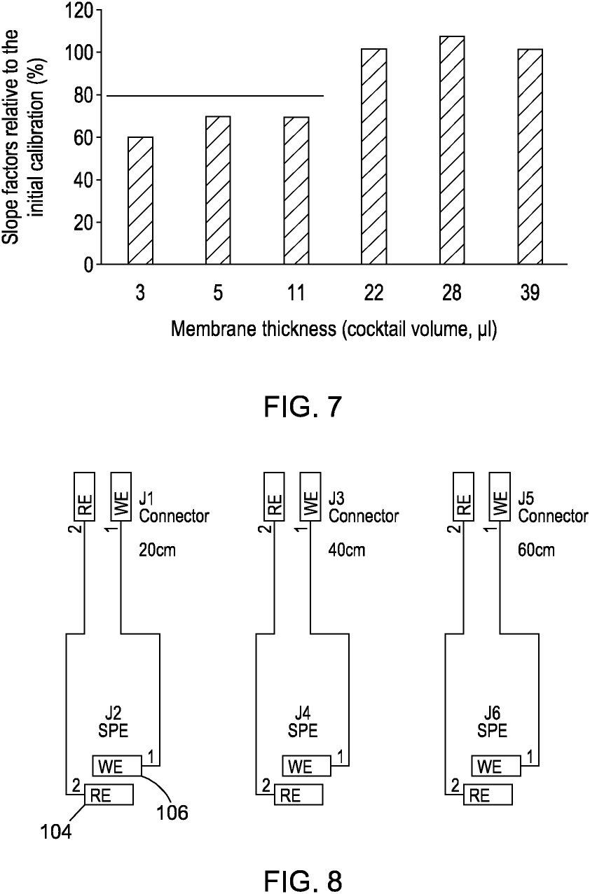

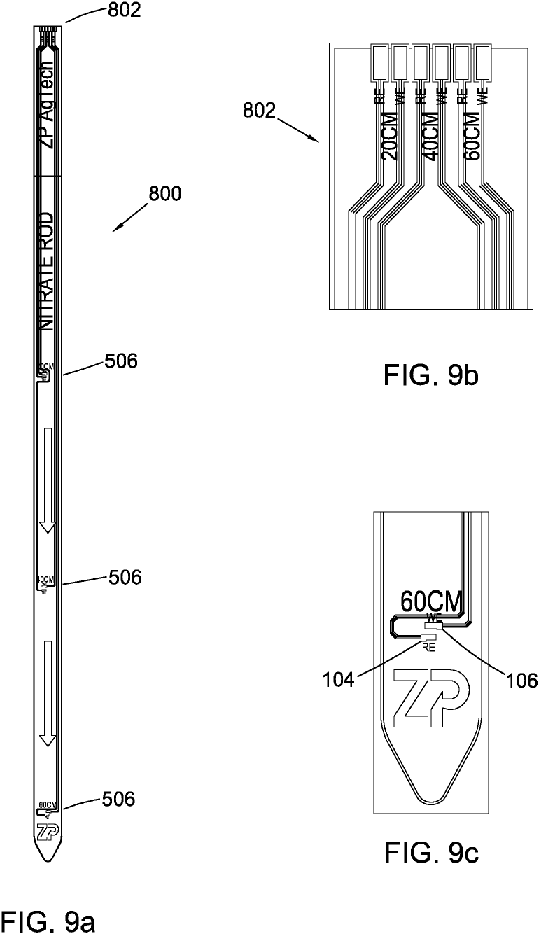

FIG. 3 shows an example of a system 300 including a plurality of sensor blades/probes, 100a – 100n, each blade as described in this disclosure, and each coupled to a respective voltage amplifier 302a – 302n. The sensors 100a – 100n can be wired together in series or parallel. The voltage amplifier provides voltage data to a data logger 304, having storage capacity, preferably at least non-volatile storage 306. In this example, the probes are coupled to the data logger via a wired connection that is used to power the probes/amplifiers, but in other examples, such as shown in FIG. 4, a wireless connection may be employed. The data logger 304 preferably incorporates non-volatile memory 306 to store the collected data and an RF transceiver 310, for example, to communicate with a computer or mobile phone network 312, to provide a link to a remote data collection/analysis computers or computer networks or servers. The computer network may alternatively be a remote computer or server such as Cloud Computing Services, which in turn may be further communicatively coupled to an end-user device such as a mobile device, smartphone and the like. A power supply 308 for the system may comprise, for example, a rechargeable battery, optionally powered from a renewable energy source such as wind or solar power. The system shown in FIG. 3 could be implemented, for example, in the probe housing apparatus described below, and according to FIGS. 5 and 6.

FIG. 4 shows a further example of a system 300 including a plurality of sensor blades/probes, where each blade is as described in this disclosure. The plurality of sensor blades 100a - 100n are distributed spatially over an area of field. The voltage amplifiers (not shown) as illustrated in FIG. 3 are comprised within a housing containing the sensors 100a-n. The sensors are communicatively coupled via a wireless connection 404 to the data logger 304. The data logger is further wirelessly connected to a Cloud based server, or remote computer network, which is wirelessly coupled (e.g. via a standard Wi-Fi internet connection) to an end user device. Advantageously, a user responsible for fertiliser maintenance is able to receive the data relating to nutrient concentration in a continuous manner from a remote location.

FIG. 5 shows an example structure of a modular probe housing 500, which is used to insert multiple sensor blades into a soil surface at a plurality of different depths. The probe housing contains a top cover, 502, at least one pole segment 503, at least one break/attachment point 504 used to connect the pole segments, at least one sensor compartments 506, and a tapered end 508 to allow insertion into soil. Advantageously, a plurality of segments 503 can be connected such that the nutrient content of soil can be probed at a plurality of depths. The pole segments can be made in a variety of lengths, such that the overall length of the probe housing 500 is adjustable. For example, the pole segments can be made into 10 cm, 20 cm, 30 cm or 40 cm lengths, such that soil nutrients can be simultaneously sensed at depths of for example 10 cm, 20 cm, 30 cm, 40 cm or 20 cm, 40 cm and 60 cm or 30 cm, 60 cm, 90 cm (the list is not exhaustive). The break attachment points may comprise a threaded connection, for example.

FIG. 6 illustrates a detailed example of the probe housing 500 as shown in FIG. 5. The probe housing 500 is capable of measuring soil chemistry at three different depths below the soil surface. The probe 500 is formed of three sensor sections: 600a, 600b, and a tip sensor section 604. The sections are connected together by break points 504. The first sensor section 600a is used to sense soil chemistry at a depth A in the soil structure. As shown, the tube portion 503 of the first sensor section 600a is sealed by a top cover 502, which may comprise a rubber stopper or plastic cover, to prevent water/soil water from entering probe 500 from the top. The sensor part 506 of the first sensor section 600a is coupled to the tube part 503 of the second sensor section 600b. The second sensor section 600b is capable of measuring soil chemistry at a depth B in the soil structure. The sensor part 506 of second sensor section 600b is coupled to the tube part 503 of tip sensor section 600c. The tip sensor section 600c is capable of measuring soil nutrients at a depth C in the soil structure. The pointed tip 508 forms the tip of probe 500. The pointed tip 508 may be a sharp metal tip which helps insertion of the probe 500 into a soil structure.

A nutrient sensor 100 is located within each sensor 506 part in a substantially horizontal orientation. The sensor compartments 506 may comprises a porous membrane 602, or simply an opening, behind which is the soil chemistry sensor 500. Electrolytes/ions in the soil directly outside of the housing membrane/opening 602 flow into the housing, enabling the soil chemistry sensor 100 to measure the soil nutrients. This is beneficial as it is possible to prolong the sensor lifetime by sensing nutrients via housing membrane/opening 602 rather than by direct contact with the soil. Alternatively, as seen in the second pole segment 600b, the sensor compartment may directly expose the membranes 104, 106 of the sensor 100 to the soil. Wiring 606 from each ion-selective electrode 106 and reference electrode 104 exits each sensor compartment 506 through a seal (not shown) to prevent leakage. In embodiments, the sensor part 506 may not extend across the full width of the probe 500, such that wiring 606 from each electrode may run up through the probe 500 in a space not occupied by the sensor parts.

Nevertheless, it will be appreciated that the structure in FIG. 6 is merely by way of example, and that the modularity of the segments allows for probe housings 500s of different lengths to be provided, with three, four, or more sensors.

### Example Fabrication of Reference Membrane

The following describes a general composition and procedure used to make a reference membrane by solvent-casting. The reference membrane formulation described below is suitable for use in conjunction with a nitrate-selective membrane as described below, e.g., in a nitrate sensor.

The mixture forming the reference membrane contain several organic, which include at least one polymer, for example poly-vinyl chloride (PVC), which is cast using a suitable solvent. More than one polymer may be used to form the reference membrane. These polymer components are dissolved in a suitable organic solvent, preferably a solvent which is not volatile at room temperature. The formulation is designed to allow the functional material to be readily applied to the electrode. The formulation has a volatile component (i.e., the solvent) that can be removed as part of curing the sensor membrane. The formulation contains polymeric material that is intended to bind the components of the membrane together.

The mixture can be vortexed in order to help the formation of a solution. A greater proportion of solvent can optionally be used, relative to the other reagents, without affecting the composition of the resulting membrane. However, a greater proportion of solvent will prolong the solvent casting process (i.e., the time taken for the solvent evaporate). Alternatively, a lower proportion of solvent may be used in order to yield a more viscous membrane-forming solution, which can aid in disposing a relatively thicker layer in a single solvent-casting step.

Solvents with a relatively high boiling point, for examples ketones, are advantageous because their relatively low volatility at room temperature provides a more gradual evaporation. In other words, it is preferred to use solvents that are not volatile at room temperature. This is particularly beneficial in screen-printing applications, where a faster-drying solvent may cause printing nozzles to clog (i.e., as a result of viscous membrane-forming solution). However, in general, many other suitable organic solvent may be used for the solvent-casting process described herein, for example, tetra-hydro furan (THF). When solvent-casting via manually pipetting, for example, a faster-drying solvent such as THF may indeed be a beneficial choice.

After a solution is formed, a deliquescent substance (i.e. hygroscopic, or moisture absorbing) may optionally be added, in excess (for example, over twice the mass of the total solution), in order to absorb any moisture present in the solution. The solution and deliquescent is preferably vortexed in order to ensure all moisture has been sequestered from the membrane-forming solution. The solution is then centrifuged, or filtered, in order to extract the remaining deliquescent material, which is in suspension in the mixture. The skilled person will appreciate that many deliquescent substances are suitable, for example, CaCl₂.2H₂O, anhydrous calcium chloride, or magnesium chloride and the like.

### Example Fabrication of Nitrate-selective Membrane

The following describes a general composition and procedure used to make a nitrate-selective membrane by solvent-casting. The following nitrate-selective membrane formulation is suitable for use with the reference membrane formulation as described above.

The organic components of the nitrate-sensing membrane comprise at least one polymer, a plasticiser, and an ionophore (i.e., a nitrate-selective ion). Suitable plasticisers include alkyl ethers, for example. Suitable polymers include example poly-vinyl chloride (PVC), however, any number of other suitable polymers may also occur to the skilled person. Moreover, a combination of more than one polymer can be used in the nitrate-selective membrane formulation. The formulation contains polymeric material that is intended to bind the ionophore (i.e., the nitrate-selective ion reagent) to the electrode, where the ionophore provides the sensing membrane with its specificity towards the ion/nutrient of interest.

For example, Tridodecylmethylammonium (TDDMA) nitrate can be used as the nitrate-selective ion. Nevertheless, it will be appreciated that additional or alternative nitrate-selective components may be employed. The function of an alkyl ether as a lipophilic plasticiser, however other suitable plasticizers may also be used in the place of alkyl ethers for the purpose of producing flexible ion-selective membranes .

The role of the plasticiser, in this sensing membrane and the membranes in general is to reduce the rigidity of the sensor’s membrane. It is generally advantageous to fabricate flexible or ‘plastic’ membranes. This is because a relatively high flexibility or plasticity increases the resilience and lifetime of the sensor, which can undergo repeated strain/bending in use. Furthermore, in examples where the base substrate is fabricated from polyethylene terephthalate it may be deformable; thus, is it advantageous to match the flexibility of the membranes to the base in order to preserve the integrity of the membranes.

As with the reference membrane, the skilled person would be able to select other suitable organic solvent for the solvent-casting process; THF is suitable, for example. A greater amount of solvent can optionally be used, relative to the other reagents, without affecting the composition of the resulting membrane. However, a greater amount of solvent will prolong the solvent casting process (i.e., the time taken for the solvent evaporate).

Once the solvent is added, the mixture can be vortexed in order to help dissolve all the components in the solvent to produce a solution.

In the same manner as for the reference membrane formulation, a deliquescent may be added in excess to the nitrate sensing formulation, once in solution, to remove any moisture present in the nitrate sensing formulation.

### Example Solvent-casting of Nitrate Sensor

Prior to solvent-casting, a substrate according to FIG. 1 is provided, where the electrodes 104, 106 are bare and not yet covered by any membrane.

0.3 µL of the nitrate sensing solution is pipetted onto the working electrode, and the same volume of reference solution is pipetted onto the reference electrode. The pipetting may be done manually, by mass-production, screen-printing, or with additive manufacturing equipment. The solutions are left to dry for at least 5 minutes, or until the solvent has dried completely and a solid-phase membrane remains. Optionally, the pipetting and drying steps can be repeated in succession on top of previously-dried membranes, to form a plurality of layers of each membrane.

The sensor may subsequently be stored, e.g. for around 2 weeks, before use, to allow any remaining solvent to dry and to ensure that the membranes are integrally and/or robustly formed. Accelerated drying can be achieved by using active methods including vacuums, desiccators, and heating.

### Alternate Nutrient-Selective Compositions

Consistent with the above formulations for the reference and nitrate-specific membranes, the same formulation may be used with the exception that the ion-selective reagent used in the nitrate-sensing membrane may be replaced by a different ion-selective reagent in order to sense other soil nutrients (or contaminants). Further consistent with the above examples, any suitable organic solvent may be used for the solvent-casting process. Relatively higher boiling point solvents, e.g. which have a higher boiling point than water, are an advantageous choice for screen-printing applications because of its more gradual rate of evaporation.

As mentioned above, it is preferred that the formulation used for the reference membrane is invariant to the nutrient of interest.

Formulations for various different nutrient-specific membranes are shown below in Tables 1 to 5 (absent the solvent used for the solvent-casting). It will nevertheless be appreciated that other sensor-membrane formulations may be used to measure other soil nutrients, including but not limited to: pH (protons), and soil contaminants including cadmium, lead and copper.

TABLE 1

| Phosphate sensing formulation component | Amount (w/w %) |
|---|---|
| Phosphate sensor as described in Carey CM & Riggan WB Anal Chem. 1994 Nov 1;66(21):3587-91; a cyclic polyamine ionophore | 6 |
| 2-nitrophenyl octyl ether (plasticiser) | 65 |
| Methyltriphenylphosphonium (additive) | 1 |
| PVC matrix | 23 |
| nitrocellulose | 5 |

TABLE 2

| Potassium sensing formulation component | Amount (w/w %) |
|---|---|
| valinomycin | 5 |
| 1,2 dimethyl-3-nitrobenzene (plasticiser) | 93 |
| Potassium tetrakis (4-Chlorophenyl) borate (additive) | 2 |

TABLE 3

| Ammonium sensing formulation component | Amount (w/w %) |
|---|---|
| nonactin | 5 |
| 2-nitrophenyl octyl ether (plasticiser) | 64 |
| Potassium tetrakis (4-Chlorophenyl) borate (additive) | 1 |
| PVC matrix | 30 |

TABLE 4

| Calcium sensing formulation component | Amount (w/w %) |
|---|---|
| Sigma-Aldrich calcium ionophore II | 1 |
| (product number 21193). | |
| 2-nitrophenyl octyl ether(plasticiser) | 65.6 |
| Potassium tetrakis (4-Chlorophenyl) borate (additive) | 0.6 |
| PVC matrix | 32.8 |

TABLE 5

| Sodium sensing formulation component | Amount (w/w %) |
|---|---|
| Sodium ionophore (e.g. as described Carden et al. 2001 (J. Exp. Bot. 52: 1353)) | 1 |
| Bis (1-butylpentyl) adipate (plasticiser) | 65.9 |
| Potassium tetrakis (4-Chlorophenyl) borate (additive) | 0.6 |
| PVC matrix | 33 |

### Additional Examples

In some examples, it is possible to tailor the thickness of the membrane layer as part of the solvent-casting process to improve the longevity and long-term sensitivity of sensors.

FIG. 7 is a graph showing results that measure the sensitivity to nitrate dependent on membrane thickness, after a prolonged use in soil. The graph shows sensitivity results for six sensors, each having a different membrane thickness, where the sensitivity has been measured before and after each sensor has been stored for one month in soil; the graph plots the ratio of these two sensitivity measurements. For each sensor, the membrane thickness shown relates to the thickness of membranes disposed on both the reference electrode and working (e.g., nitrate-selective) electrode. The thickness is defined here by the amount of cocktail (e.g., precursor mixture used in the solvent-casting process) used to form both of the nitrate-selective and reference electrode membranes by solvent casting.

Six solid-state nitrate sensors were calibrated in nitrate solutions (initial calibration) before and after one month of storage buried in soil. The calibration was performed by recording voltages (V) in different nitrate solutions, which were fitted to the nitrate calibration (as pNO₃ ₌ – log₁₀[NO₃⁻]) with a linear regression line. The line slope factor (i.e. the gradient) b, in the formula V ₌ a + b*pNOs indicates the response of the sensors to the change in nitrate concentration (e.g. the sensitivity). The ratio of the line slope factors calculated for each sensor before and after being buried in soil for a month are shown in FIG. 7.

Generally, it can be seen that sensors with greater thickness (e.g., those sensors having membranes prepared with a cocktail volume of 22, 28, and 29 µL) have a ratio of gradients at or close to 100%. This corresponds to a sensor whose sensitivity to nitrate is substantially or completely unchanged even after having been buried in soil for one month. These data thus show that increasing the membrane thickness can improve the ability of a sensor to maintain sensitivity to nitrate after prolonged use. This is important and advantageous for the quality and lifetime of the sensors when used in soil.

FIGS. 9 shows a further example of a soil probe 800 comprising multiple sensors for inserting into surface at a plurality of depths. This example shows a probe where the circuitry required for all sensors is disposed on a printed circuit board (PCB). FIG. 8 outlines the circuitry of the three sensors used on the PCB of the probe of FIGS. 9. The three sensors are of the same design with respect to the arrangement of the working and reference electrodes, and are disposed at 20 cm, 40 cm, and 60 positions, e.g. for use at those depths when the probe 800 is placed in soil.

FIG. 9a shows the full length of the PCB soil probe 800, having three sensor locations 506 spaced 20 cm apart, e.g. to detect nitrate at 20, 40, and 60 cm depth when placed in soil. FIG. 9b shows the top portion 802 of the probe, onto which is printed the electrical connections for each of the three sensors (e.g., corresponding to electrical contacts 102). FIG. 9c shows the base / distal end of the probe, which comprises a reference electrode 104 and working electrode 106 at the 60 cm position. Before use, the reference and sensing membrane, respectively, may be solvent-cast onto the reference and working (e.g., nitrate-sensitive) electrode locations. The configuration of the electrodes of the other two sensors at the 20 and 40 cm position is the same as the 60 cm sensor.

No doubt many other effective alternatives will occur to the skilled person. For example, examples of the sensor are not restricted to being used in-situ. For example, a core sample may be extracted and mixed with water, and subsequently brought to a sensor for measurement. Similarly, examples described above may be employed to measure nitrate levels in leaf sap, or leaves (e.g. leaves having been pulverised and mixed with water), which has value in research and breeding as well as in farm crop testing.

It will be understood that above description is not an exhaustive list of examples and embodiments and the invention, and is intended to encompass modifications apparent to those skilled in the art and lying within the scope of the claims appended hereto.

1. A solid-state soil nitrate sensor, comprising:
a sensor blade for inserting into the soil, the sensor blade comprising an electrically insulating substrate;
first and second electrodes disposed on the substrate, each electrode comprising:
a sensing region located towards an end of the sensor blade inserted into the soil,
a contact region displaced away from the end of the sensor blade and electrically connected to the sensing region, for making an electrical connection to the electrode;
electrical insulation over each of the first and second electrodes between the sensing region and the contact region;
a reference membrane over the sensing region of the electrode; and
a nitrate sensing membrane over the sensing region of the second electrode;
wherein the sensing regions of the first and second electrodes are less than 10 mm apart on the sensor blade; and
wherein the reference membrane and the nitrate sensing membrane each comprise one or more layers of solvent-cast polymer.

2. A solid-state soil nitrate sensor as claimed in claim 1 wherein the nitrate sensing membrane comprises a nitrate-ion selective membrane.

3. A solid-state soil nitrate sensor as claimed in claim 1 wherein the nitrate sensing membrane comprises a nitrate-sensing reagent to convert nitrate ions to electrons.

4. A solid-state soil nitrate sensor as claimed in claim 2 wherein the nitrate sensing membrane comprises two layers of solvent-cast polymer, an inner layer comprising the nitrate-ion selective membrane or nitrate-sensing reagent, and an outer less-selective layer, wherein the outer less-selective layer is harder than the inner layer.

5. A solid-state soil nitrate sensor as claimed in claim 1 wherein the reference membrane comprises a charge-selective membrane which allows positive charge to flow through the reference membrane and inhibits movement of chloride ions through the reference membrane.

6. A solid-state soil nitrate sensor as claimed in claim 5 wherein the reference membrane comprises two layers of solvent-cast polymer, an inner layer comprising the charge-selective membrane, and an outer less-selective layer, wherein the outer less-selective layer is harder than the inner layer.

7. The solid-state soil nitrate sensor according to claim 1 wherein the first electrode is formed from any one of: carbon, sliver chloride, gold, or platinum.

8. The solid-state soil nitrate sensor according to claim 1 wherein the first and second electrodes are first and second carbon or silver chloride electrodes.

9. (canceled)

10. (canceled)

11. (canceled)

12. (canceled)

13. (canceled)

14. A solid-state sensor for in-situ sensing of soil nutrients, the solid-state sensor comprising:
an electrically insulating substrate bearing a first and second electrically conductive track;
a contact region defined by electrical contacts at an end of each electrically conductive track;
a sensing membrane disposed over a second end first electrically conductive track, defining an ion-selective electrode;
a reference membrane disposed over a second end of the second electrically conductive track defining a reference electrode;
an electrically insulating cover disposed over the first and second electrically conductive tracks;
wherein each of the sensing membrane and the reference membrane comprises a layer of solvent cast polymer, and wherein the sensing membrane comprises a sensing reagent for sensing a specific nutrient; and
wherein a separation between the second end of each electrically conductive track is less than around 10 mm.

15. A solid-state sensor as claimed in claim 14, wherein the sensing reagent is for sensing a concentration of nitrate.

16. A solid-state sensor as claimed in claim 14, wherein each of the sensing membrane and the reference membrane comprises two layers of solvent-cast polymer.

17. A solid-state sensor as claimed in claim 14, wherein the substrate comprises plastic or ceramic.

18. A solid-state sensor as claimed in claim 14, wherein each of the first and second electrically conductive tracks comprise carbon or silver chloride.

19. A solid-state sensor as claimed in claim 14, wherein substantially all of the first and second electrically conductive tracks are covered by the electrically insulating cover, and wherein the electrically insulating cover provides waterproofing.

20. A solid-state sensor as claimed in claim 14, wherein each of the first and second electrically conductive tracks are formed by screen-printing onto the substrate.

21. A solid-state sensor as claimed in claim 14, wherein the electrical contacts are integrally formed as part of the first and second electrically conductive tracks.

22. A solid-state sensor as claimed in claim 14, wherein each of the sensing membrane and the reference membrane contain the same polymer, preferably high molecular weight polyvinylchloride (PVC).

23. A solid-state sensor as claimed in claim 14 further comprising a voltage sensor coupled to the contact region, and a wireless network transmitter coupled to said voltage sensor to enable wireless collection of soil chemistry data from said soil chemistry sensor.

24. A solid-state sensor as claimed in claim 1, wherein the electrical contacts or contact regions are suitable for coupling with an external data logging device.

25. A solid-state sensor as claimed in claim 24, wherein the external data logging device is configured to measure an impedance value indicative of a concentration of nutrient in a soil sample.

26. (canceled)

27. A system for continuous in-situ sensing of soil nutrients comprising a plurality of solid-state sensors as defined in claim 1, wherein the system further comprises:
a plurality of signal amplifiers, one for each solid-state sensor, wherein each signal amplifier is communicatively coupled to a data logger for receiving signals from each solid-state sensor.
